(19) United States
(12) Patent Application Publication
(10) Pub. No.: US 2024/0024525 A1
(43) Pub. Date: Jan. 25, 2024

(54) UV Port Disinfection System and Method

(71) Applicant: Northstar Medical Technologies, LLC, Beloit, WI (US)

(72) Inventors: Alec Rosenberg, Madison, WI (US); Michael Allex, Verona, WI (US); Walter Brumm, Fitchburg, WI (US); Raymond Heasty, McFarland, WI (US); Eric Huerth, Sauk City, WI (US)

(73) Assignee: Northstar Medical Technologies, LLC, Beloit, WI (US)

(21) Appl. No.: 18/223,504

(22) Filed: Jul. 18, 2023

Related U.S. Application Data
(60) Provisional application No. 63/368,843, filed on Jul. 19, 2022.

(51) Int. Cl. A61L 2/10; A61L 2/24

(52) U.S. Cl. CPC A61L 2/10; A61L 2/24; A61L 2202/11; A61L 2202/14; A61L 2202/24

(57) ABSTRACT

A UV port disinfection system and method is disclosed. The UV port disinfection system can include a UV light source, a UV arm, and a UV arm pivoting axis, wherein the UV light source is connected to the UV arm, and wherein the UV arm is located on the UV arm pivoting axis and is rotatable on the axis to allow the UV arm to extend to one or more fluid input ports. Further, when the arm is over said fluid input port, said UV light source activates and emits UV light which disinfects said port. In a further embodiment, the UV port disinfection system can include a controller. A method for UV port disinfection is also disclosed.

### CROSS-REFERENCE TO RELATED APPLICATION

This application claims the benefit of the filing date of U.S. provisional application Ser. No. 63/368,843, filed Jul. 19, 2022, entitled, “UV Disinfection of Ports On An Aseptic Fluid Handling System,” which is hereby incorporated by reference as if fully set forth herein.

### FIELD OF THE INVENTION

This disclosure generally relates to fluid handling systems. In particular, the disclosure is directed to systems with ports for introducing fluids into the system and is particularly directed to reducing contaminants such as microorganisms being introduced to the system through the ports. A particular application is for fluid handling systems for radiopharmaceutical products, particularly nuclear medicine, and, more particularly, systems and methods of processing radioactive nuclides.

### BACKGROUND OF THE INVENTION

In general, fluid handling systems have ports for introducing fluids into the system. One of the concerns with such fluid intake ports is the prevention of microbes, such as bacteria and fungi, from contaminating the fluid intake ports. Contamination of the ports can then contaminate the fluids being introduced into and used in the system rendering the fluids unsafe and unsatisfactory for their intended purposes.

Such fluid contamination is a particular concern wherein the fluids are intended for introduction to the human body. If contaminants reach an unsafe level, it can cause serious health concerns in humans and possibly chronic diseases.

As a result, disinfection of ports for the purpose of aseptic connection is necessary to minimize the introduction of microbes, and other contaminates into a fluid system. A common method for disinfecting ports is the use of disinfecting wipes. The wipes can be provided in the form of alcohol wipes, hydrogen peroxide wipes, or the like. The wipes can be used to clean the port before connecting the ports to a fluid container and before the introduction of fluids. Once the connection between the port fluid container is removed, the port can be capped. The port can be disinfected after the connection is broken. Alternatively, or in addition to, the port can be disinfection at a later time when the connection is reestablished.

Such wipes, however, generate waste. Further, the wipes can be contaminated with microbes after wiping the port. The wipes can also be contaminated with radioactive material (RAM) and thus need to be disposed of as radioactive waste. These microbes and contaminants can then contaminate the user or operator of the fluid handling system and can also contaminate the environment. Further, if the wipes are not effective in cleaning the port, microbes or contaminants can get into the patient.

Moreover, in cases where radioactive fluids are being introduced or used in a fluid system, the resulting wipes may become radioactive after wiping the port. Such wipes are hazardous to the user and environment, and disposal of such wipes can be dangerous and expensive. Accordingly, safe and proper disposal of the wipes, especially the radioactive ones, is required.

Disposal of such wipes, however, can be complex and potentially expensive. Consequently, there is a need for safe and proper disinfection of such ports for a fluid handling system. Preferably such disinfection is also cost-efficient.

Waste production, as well as the complexities associated with radioactive waste disposal, can be eliminated or minimized with the use of an ultraviolet light port disinfection system. Ultraviolet (UV) light is an established approach to killing potentially harmful microbes, but the implementation of a UV port disinfection system for ports on an aseptic fill fluid handling system is not known to have been previously demonstrated.

### BRIEF SUMMARY OF THE INVENTION

The present invention contemplates a UV port disinfection system and assembly for the prevention of microbes, such as bacteria and fungi, and other microorganisms from contaminating fluid intake ports. In an exemplary embodiment, the UV port disinfection system and assembly is on an aseptic fluid handling system.

An exemplary embodiment is directed to a UV port disinfection system comprising a UV light source, a UV arm, and a UV arm pivoting axis, wherein the UV light source is connected to the UV arm, and wherein the UV arm is located on the UV arm pivoting axis and is rotatable on said axis to allow said UV arm to extend to one or more fluid input ports of a fluid handling system. Further, when said UV arm is over said port, said UV light source activates and emits UV light which disinfects said port.

In some embodiments, the UV light source is activated for a predetermined period of time

In a further exemplary embodiment, a UV port disinfection system includes a UV light, a UV arm, and a controller. UV light is housed in or on the UV arm, which can be positioned with a line of sight to the fluid contact surfaces of a fluid port. The controller can detect whether the UV arm is positioned appropriately to allow the UV light to be positioned with direct sight of the port's fluid contact surfaces. When the UV light is positioned in sight of the port, the controller can activate the light to kill microbes on the fluid port surfaces.

While activating the light, the current/voltage of the UV light can be measured, and the controller can infer the intensity of the light output based on these measurements and using data sheets so as to be consistent and effective microbial kill. The amount of intensity of UV light can depend on the parameters, and the controller can adjusts accordingly. Detection can occur via UV light sensor, current detection, voltage and wattage sensing, or some other means. In a further embodiment, the home position can include a UV detector which could be used to spot check the LED.

Accordingly, in some exemplary embodiments, the UV port disinfection system comprises at least one of a current detector designed to detect an amount of a current drawn by the UV light source and a temperature detector designed to detect a temperature rise on the UV light source. Further, in some embodiment, the controller is designed to determine the UV light source is activated when at least one of the current drawn by the UV light source and the temperature rise on the UV light source are greater than a predetermined threshold. In some embodiments, the controller is designed to prevent operation of the UV port disinfection system when the controller determines that the UV light source is not activated.

Another exemplary embodiment is directed to a port disinfection system comprising a movable arm comprising a pivoting axis; a plurality of light sources connected to the movable arm; and a plurality of fluid input ports, wherein the movable arm is designed to pivot about the pivoting axis so that in a first position, at least one light source of the plurality of light sources is positioned over at least one fluid input port of the plurality of fluid input ports, wherein when the at least one light source is positioned over the at least one fluid input port, the at least one light source activates and emits light which disinfects the least one fluid input port, and wherein when each light source of the plurality of light sources are not activated, the movable arm is positioned at a home position.

In a further exemplary embodiment, the movable arm is designed to pivot about the pivoting axis so that in a second position, a first light source of the plurality of light sources is positioned over a first fluid input port of the plurality of fluid input ports, and a second light source of the plurality of light sources is positioned over a second fluid input port of the plurality of fluid input ports.

In a further exemplary embodiment, each of the plurality of light sources are independent selected from the group consisting of a UV LED light source, a mercury UV light source, and a high intensity visible light flash source.

Another exemplary embodiment is directed to a method for disinfecting one or more fluid ports utilizing a UV port disinfection system with a UV arm and a UV light source located on the UV arm. The method comprises moving the UV arm over one input port, when the UV arm is over the input port, activating the UV light source, and emitting UV light from the UV light source and disinfecting the port.

The UV port disinfection system and method of the present invention is advantageous as it mitigates the risks previously associated with the use of automated ultraviolet light—including inconsistent or incomplete microbial kill—by detecting the position and electrical current, voltage, wattage, and LED temperature for the UV light applied to a surface.

### BRIEF DESCRIPTION OF THE DRAWINGS

In the drawings forming a part of this disclosure,

FIG. 1 is a diagram illustrating an examplary embodiment of an assembly of an ultraviolet (UV) port disinfection system from a user-facing view;

FIG. 2 is a diagram illustrating the reverse or underneath side of an exemplary embodiment of assembly of FIG. 1.

FIG. 3 is a diagram illustrating cross-sectional view of an exemplary embodiment of assembly of FIG. 1.

FIG. 4 is a diagram illustrating in an exploded view an exemplary embodiment of elements that can form a UV arm.

FIG. 5 is a diagram illustrating in an exploded view an exemplary embodiment of limit switch and detent plate.

FIG. 6 shows an illustration of a cross sectional view of an exemplary embodiment of a connection of a detent plate with a detent arm of an assembly of FIG. 3.

FIG. 7 is block diagram for an exemplary example of UV disinfection position detection.

### DETAILED DESCRIPTION OF AN ILLUSTRATED EMBODIMENT

Although this invention is susceptible of embodiments in many different forms, that are shown in the drawings and will be described in detail herein in specific embodiments with the understanding that the present disclosure is an exemplification of the principles of the invention. It is not intended to limit the invention to the specific illustrated embodiments. The features of the invention disclosed herein in the description, drawings, and claims can be significant, both individually and in any desired combinations, for the operation of the invention in its various embodiments. Features from one embodiment can be used in other embodiments of the invention.

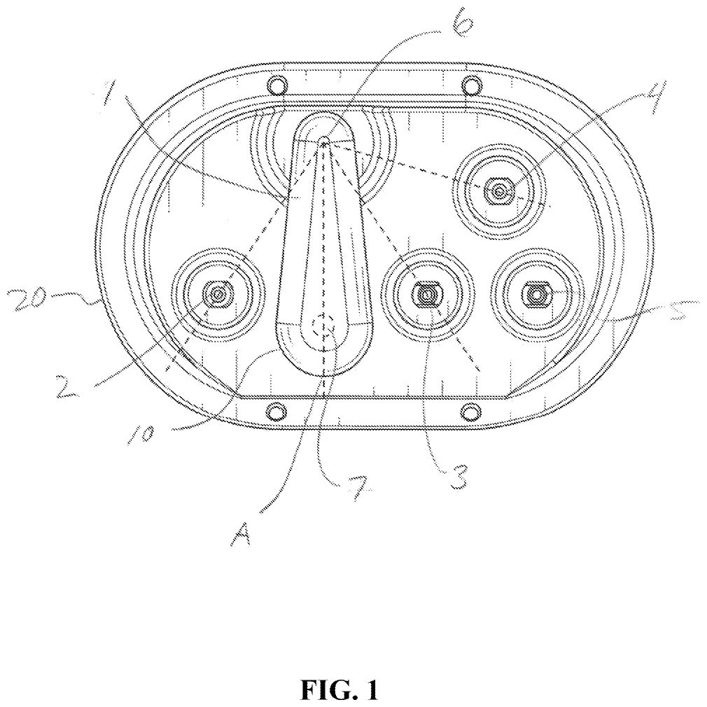

Referring to FIG. 1, an examplary embodiment of an assembly 10 of an ultraviolet (UV) port disinfection system is shown. The assembly 10 can include a UV arm 1 and a UV arm pivot point/axis 6. In an examplary embodiment, the UV arm can be made of stainless steel. Other assembly components can be made of stainless or plastic, such as a cast urethane. Reference number 7 shows one possible location at the end of the UV arm of a UV light source, which is part of assembly 10 and located on the underside of UV arm 1 and from which UV light is emitted in a direction away from the UV arm 1. The UV light is preferably on the opposite end of the UV arm 1 from the UV arm pivot/axis 6. The UV disinfection system and assembly could be located in a connection port area 20, such as a reagent connection port area for a system for preparing and processing radiopharmaceutical products, particularly for nuclear medicine and more particularly, for processing radioactive nuclides.

In one embodiment, the UV arm 1 is capable of being rotated on pivot point/axis 6 to reach one or more input ports. The one or more input ports could be, for example, a NaOH input port 2, a saline input port 3, a sterile water input port 4, and an ozonated water output port 5. The ports shown herein are illustrative, as more or less, and/or different ports, for this and different purposes and objectives, are contemplated within the scope of this description. In a further embodiment, when not located over a port, the UV arm 1 can be located in a storage spot or a home position A.

The assembly 10, connection port area 20, and UV arm 1 are shown in a user-facing view in FIG. 1. The assembly allows the user to move the UV light, which is located on the underside of the UV arm 1 at end 7, from a storage spot or home A to directly over one or more input ports, such as for example, ports 2, 3, 4 and 5 shown in FIG. 1. In this embodiment, the UV arm 1 pivots about the UV arm pivot point/axis 6 so that end 7 can be located over one or more ports. There can also be a detent stop position over each of the ports 2, 3, 4, and 5 that the end 7 of UV arm 1 can reach. In FIG. 1, the UV arm 1 is shown in a home position A where the end 7 and UV light source are not positioned over any port.

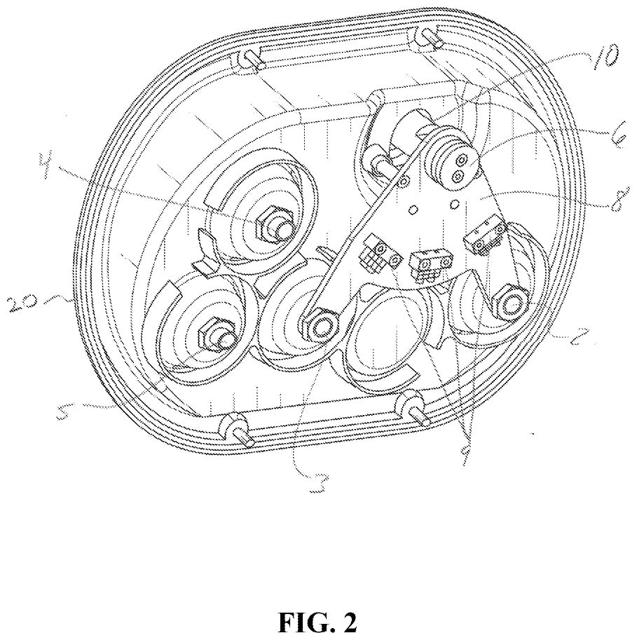

FIG. 2 shows the reverse or underneath side of assembly 10 and connection port area 20 of FIG. 1. In particular, the reverse or underside of input ports 2, 3, 4, and 5 are shown. Further, the reverse or underside of the UV arm pivot point/axis 6 is also shown. This view is typically not user facing.

FIG. 2 shows a further embodiment of the assembly having a detent plate 8 and limit switches 9. The detent plate 8 can limit the UV arm pivot point/axis 6 so that in this embodiment, the UV arm 1 can only reach input port 2 and input port 3. It is contemplated that a similar detent plate 8 can be used to limit the reach of UV arm 1 to any one or more of the ports in connection port area 20. In one examplary embodiment, the UV arm can cover multiple ports simultaneously.

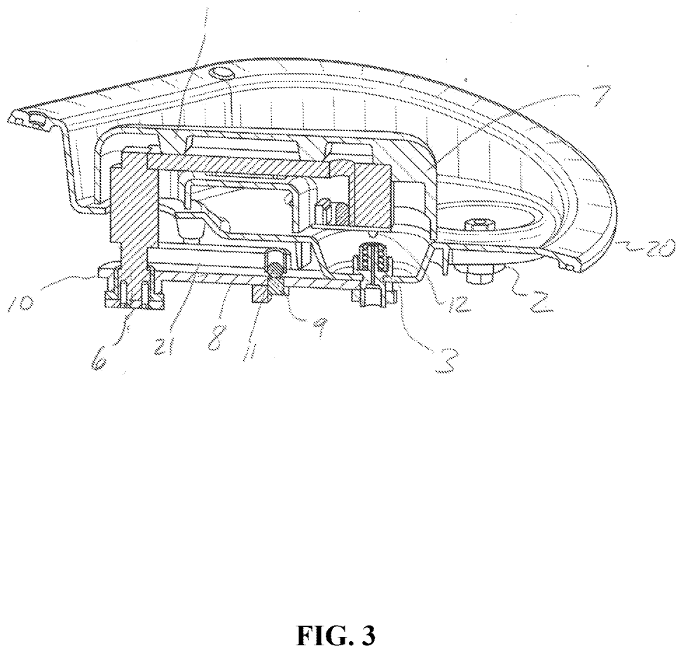

FIG. 3 shows a cross-sectional view of an exemplary embodiment of assembly 10 of FIG. 1. FIG. 3 shows a UV light source 12 which can be located at end 7 on the UV arm 1. The UV light source can use a known UV light source. The UV light source can be any source which is known in the art. For example, the light source could be a UV LED or a mercury UV. Alternate light sources can include very high-intensity visible light flash sources. In the exemplary configuration illustrated in FIG. 3, the UV arm 1 is positioned directly over port 3. The UV arm 1 is held in this spot by the detent plate 8 and the limit switch 9.

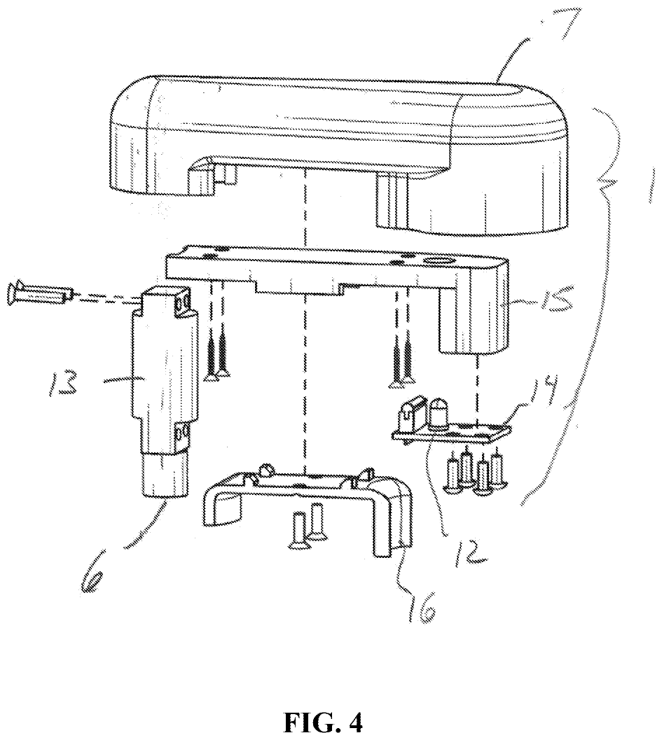

As shown in the embodiment of FIG. 3, a detent arm 21 can be rigidly connected to the UV arm 1 through structural members. These are typically machined stainless steel or metal components having the strength for carrying or supporting another component. For example, in one non-limiting embodiment, the detent arm 21 can also act as a structural member. A more detailed discussion of these structural members appears below with reference to FIG. 4. A ball-spring plunger 11 can be attached to the detent arm 21. The ball-spring plunger 11 in this embodiment is shown in a detent position within the detent arm 21. In action in this embodiment, the ball-spring plunger 11 contacts a limit switch 9 when the UV light is positioned directly over one of the ports, which in this embodiment is the port 3, to confirm that the light is appropriately positioned over the port. From there, the UV light source can be activated, and the UV light issued therefrom will disinfect and/or sterilize the port.

FIG. 4 shows, in an exploded or dissembled view, an illustration of an exemplary embodiment of elements or parts that can form or be included in UV arm 1. As shown in FIG. 4, assembly arm 1 can include a UV light printed circuit board assembly (PCBA) 14, using a known PCBA, which can be rigidly attached to a UV arm bar 15. UV arm bar 15 can be rigidly attached to a UV arm pivot shaft 13 which is part of the UV arm pivot/axis 6. UV arm pivot shaft 13 can pivot on pivot point/axis 6 for moving UV arm 1 from the home position to one or more ports 2, 3, 4, 5, and back to the home position. The UV light source 12 can be mounted on the UV light PCBA and thus rotates about the UV arm pivot shaft 13 and axis 6. A UV arm bottom cover 16 and a cover 21 over pivot shaft 13 and arm bar 15 of UV arm 1 prevent direct user contact with the UV light PCBA 14, UV light from UV light source 12, and the ports.

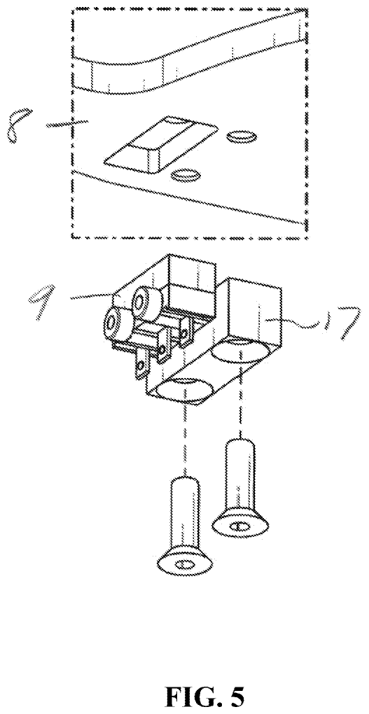

FIG. 5 shows an illustration of an exemplary embodiment of limit switch 9. Limit switch 9 can be rigidly connected to a limit switch mount 17. Limit switch mount 17 can then be rigidily mounted to the detent plate 8. In an exemplary embodiment, the mount block is threaded such that a limit switch can be fastened to the mount block. The detent plate is also threaded so the mount block can be fastened to the detent plate.

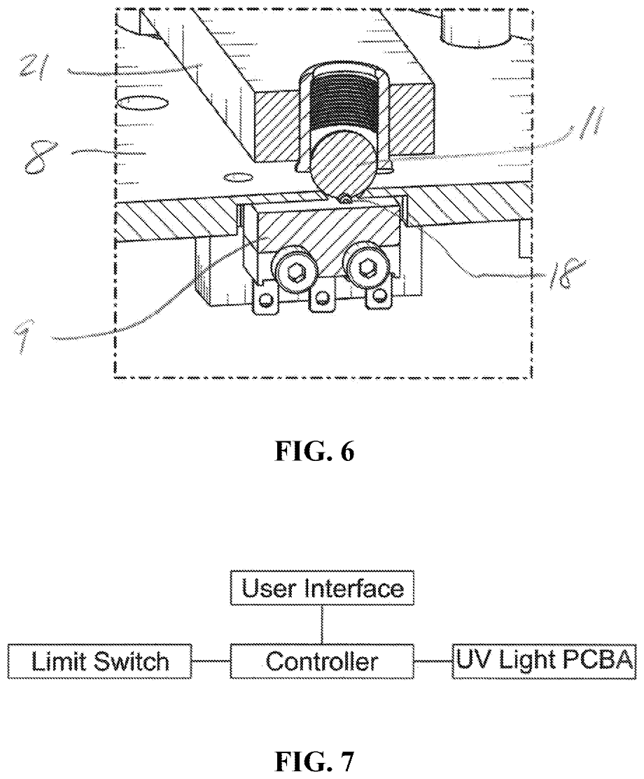

FIG. 6 shows an illustration of a cross-sectional view of an exemplary embodiment of a connection of detent plate 8 with detent arm 21. In this embodiment, the ball-spring plunger 11 is shown in cross-sectional view with a limit switch button 18. In operation, the detent arm 21 of UV arm 1 is positioned over a port (such as one of ports 2, 3, 4 or 5) such that the ball-spring plunger 11, which in one embodiment is at the end of the detent arm 21, is directly over a chamfered hole within the detent plate 8. The chamfered hole is in this embodiment associated with one of the ports. Once over the chamfered hole, the ball-spring plunger 11 depresses the limit switch button 18. When the limit switch button 18 is depressed, the UV light source 12 is directly over a port (such as port 2, 3, 4, or 5). An example is shown in FIG. 3 wherein UV arm 1 is over port 3, with ball-spring plunger 11 over a chamfered hole and limit switch 9. Thus, position detection of the UV light is achieved. Once in position, the UV arm requires a force to move the arm out of the detent position. It is the user's responsibility via instruction on the HMI of which port the arm should be positioned to.

Once over the port, UV light is dispensed from UV light source 12 and disinfects the port. Once the port has been disinfected, the UV arm can be manually moved to each position by the user. In some embodiments, the UV can be designed to automatically move to another position once the port has been disinfected.

FIG. 7 is a block diagram for an exemplary UV disinfection position detection system. An illustrative example utilizing the elements of the above embodiment is described. The embodiment is not limited to these elements. In this embodiment, a controller detects a signal when the ball-spring plunger 11 contacts the limit switch 9 via the limit switch button 18. When a user interacts with a user interface, such as on the device or a remote device, to activate UV disinfection, the controller checks via the signal from the limit switch to ensure that the UV arm is positioned such that the UV light is directly over a fluid port. When this position is confirmed, the controller then activates the UV light via the UV light source and UV light PCBA to disinfect the fluid port. If the signal indicates that the UV light is not directly over a fluid port, the controller will not activate the UV light.

The controller can be a microprocessor. Alternately, the controller can include one or more discreet transistors, op-amps, comparators, 555-timers, field programmable gate arrays (FPGA), or any combination thereof. One example could be discreet circuitry that is powered through a microswitch and activates as soon as the arm is over a switch/detent.

In an exemplary embodiment, the UV port disinfection system assembly 10 is located on an aseptic fluid handling system. The UV arm 1 is positioned over an input port, such as, a NaOH input port 2, a saline input port 3, a sterile water input port 4 or an ozonated water output port 5 by rotating UV arm 1 on UV arm pivot point/axis 6. This allows the UV light to be positioned with direct sight of the port's fluid contact surfaces. When the UV light is positioned in sight of the port, the UV light source 12 is activated to disinfect the fluid port surfaces.

In a further embodiment, position detection is integrated into an ultraviolet disinfection system by the integration of functional detection, including current and temperature detection. This integration of functional detection into the ultraviolet disinfection system effectively mitigates the risks associated with the automation of such a system for microbial kill. Further, the controller can be configured to detect the amount of current drawn by the UV light and a temperature rise on the UV light PCBA. The combination of this current and temperature detection can ensure that the UV light is activated and mitigates the risk of a burnt-out UV light. Thus is can be effectively ensured that UV Light was delivered to the port.

In a further embodiment, the ultraviolet detection system described herein can also have the ability to prevent operation in the event of a failure of the disinfection system, and/or can request user intervention in the event of a failure, and/or can notify a third party of a failure or aging. This can be done by the controller in FIG. 7. Further, when detecting a failure to provide UV light, the controller can notify the user via the user interface.

In an alternative embodiment, the system as described above, can control and detect via voltage rather than current.

Each of the patents, patent applications, and articles cited herein is incorporated by reference. The use of the article “a” or “an” is intended to include one or more.

The foregoing description and the examples are intended as illustrative and are not to be taken as limiting. Still, other variations within the spirit and scope of this invention are possible and will readily present themselves to those skilled in the art.

Specific embodiments of an assembly of an ultraviolet (UV) port disinfection system according to the present invention have been described for the purpose of illustrating the manner in which the invention can be made and used. It should be understood that the implementation of other variations and modifications of this invention and its different aspects will be apparent to one skilled in the art and that this invention is not limited by the specific embodiments described. Features described in one embodiment can be implemented in other embodiments. The subject disclosure is understood to encompass the present invention and any and all modifications, variations, or equivalents that fall within the spirit and scope of the basic underlying principles disclosed and claimed herein.

What is claimed:

1. A UV port disinfection system comprising:
a UV light source;
a UV arm; and
a UV arm pivoting axis,
wherein the UV light source is connected to the UV arm,
wherein the UV arm is located on the UV arm pivoting axis and is rotatable on the UV arm pivoting axis to allow the UV arm to extend to one or more fluid input ports, and
wherein when the UV arm is over the one or more fluid input ports, said UV light source activates and emits UV light which disinfects the one or more fluid input ports.

2. The UV port disinfection system of claim 1, wherein the UV light source is activated for a predetermined period of time.

3. The UV port disinfection system of claim 1 further comprising a detent stop position over the one or more fluid input ports.

4. The UV port disinfection system of claim 3 wherein the detent stop position comprises a detent plate, wherein the detent plate limits access of the UV to particular input ports.

5. The UV port disinfection system of claim 4 wherein the detent stop position further comprises a limit switch, wherein the limit switch is connected to the detent plate to limit access to a particular input port of the one or more fluid input ports and to limit activation of the UV light source.

6. The UV port disinfection system of claim 5 wherein the detent stop position further comprises:
a ball-spring plunger connected to a detent arm; and
a limit switch button coupled to the limit switch,
wherein when the UV arm is over one of the ports, the ball-spring plunger depresses the limit switch button of the limit switch, and
thereafter, the UV light source is activated.

7. The UV port disinfection system of claim 1 further comprising a controller.

8. The UV port disinfection system of claim 6 wherein a controller detects a signal when a user interacts with a user interface to activate UV disinfection, the controller checks via the signal from the limit switch to ensure the UV arm is positioned such that the UV light source is directly over a fluid port, when position is confirmed, the controller than activates the UV light via the UV light source, and UV light PCBA to disinfect the fluid port.

9. The UV port disinfection system of claim 6, further comprising at least one of a current detector designed to detect an amount of a current drawn by the UV light source and a temperature detector designed to detect a temperature rise on the UV light source.

10. The UV port disinfection system of claim 9, wherein the controller is designed to determine the UV light source is activated when at least one of the current drawn by the UV light source and the temperature rise on the UV light source are greater than a predetermined threshold.

11. The UV port disinfection system of claim 8, wherein the controller is designed to prevent operation of the UV port disinfection system when the controller determines that the UV light source is not activated.

12. A method for disinfecting one or more input ports utilizing a UV port disinfection system with a UV arm and a UV light source located on the UV arm, the method comprising:
moving the UV arm over a first input port,
when the UV arm is over the first input port, activating the UV light source, and
emitting UV light from the UV light source and disinfecting the first input port.

13. The method of claim 12 further comprising:
after disinfecting the first input port, the UV arm is moved to either a second port to disinfect or moved to a home position for future use.

14. The method of claim 12 further comprising:
when the UV arm is over the first input port, depressing a limit switch associated with the first input port using a ball-spring plunger located on the UV arm indicating that the UV light is directly over the first input port, and
then emitting the UV light over the first input port.

15. The method of claim 14 further comprising:
producing a signal when the UV arm is over the first input port and the ball-spring plunger depresses the limit switch; and
detecting the signal by a controller to ensure that the UV light is over the first inout port,
wherein the controller then activates the UV light.

16. A port disinfection system comprising:
a movable arm comprising a pivoting axis;
a plurality of light sources connected to the movable arm; and
a plurality of fluid input ports,
wherein the movable arm is designed to pivot about the pivoting axis so that in a first position, at least one light source of the plurality of light sources is positioned over at least one fluid input port of the plurality of fluid input ports,
wherein when the at least one light source is positioned over the at least one fluid input port, the at least one light source activates and emits light which disinfects the least one fluid input port, and
wherein when each light source of the plurality of light sources are not activated, the movable arm is positioned at a home position.

17. The port disinfection system of claim 16, wherein the movable arm is designed to pivot about the pivoting axis so that in a second position, a first light source of the plurality of light sources is positioned over a first fluid input port of the plurality of fluid input ports, and a second light source of the plurality of light sources is positioned over a second fluid input port of the plurality of fluid input ports.

18. The port disinfection system of claim 16, wherein the each of the plurality of light sources are independent selected from the group consisting of a UV LED light source, a mercury UV light source, and a high intensity visible light flash source.
